(19) United States
(12) Patent Application Publication
(10) Pub. No.: US 2008/0261309 A1
(43) Pub. Date: Oct. 23, 2008

(54) IN VITRO MULTIPLICATION OF HOODIA PLANTS

(75) Inventors: Blessed N. Okole, Kwazulu Natal (ZA); Nadine Moodley, Phoenix (ZA)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A/ UNILEVER, Englewood Cliffs, NJ (US)

(21) Appl. No.: 11/737,825

(22) Filed: Apr. 20, 2007

(51) Int. Cl. C12N 5/04

(52) U.S. Cl. 435/420

(57) ABSTRACT

A tissue culture micropropagation process for Hoodia plants, which achieves new shoots in the multiplication stage of the process by using an inventive combination of phytohormones.

### FIELD OF THE INVENTION

The present invention relates to micropropagation via tissue culture for Hoodia plants, particularly the multiplication stage of the micropropagation.

### BACKGROUND OF THE INVENTION

Hoodia genus of plants are succulent desert plants which belong to Apocynaceae family. The Apocynaceae family includes numerous other genera of plants. Hoodia plants grow predominantly in South Africa. Hoodia gordonii also grows in Botswana and Namibia. Certain actives obtainable from Hoodia plants, e.g. steroidal glycosides, have been shown to have appetite suppressant activity and to be useful in weight management products. Many of these species, e.g. Hoodia gordonii, are on the endangered list, so that collection of the wild plants is not possible. Commercial cultivation and harvesting of Hoodia plants has become of interest.

Typically, plants are reproduced by collecting and then planting the seeds. Growing Hoodia from seeds, however, takes a longer period from planting to harvesting and plants that are propagated from seeds have a higher probability of early witting and death, for instance caused by some seed and soil borne pathogens belonging to the genus (Rhizoctonia, Pythium, Phytophthora and Fusarium species). Propagation via tissue culture method allows one to start with clean, disease free materials, with a high vigor and health. Tissue culture method also achieves a shorter time from planting to harvest. Furthermore, the tissue culture method results in a more exact “cloning” of the genetic profile of a parent plant, allowing cultivation and bulking of an elite line of plants with genetic conformity, over a relatively short period of time.

WO 2006/051334 discloses cell cultures of Hoodia. Cell culture differs from tissue culture. Cell cultures seek not to propagate the plant, but to produce the eventual active (e.g., steroidal glycosides from Hoodia) directly from cultured cells, thus obviating the need for growing the plant. Tissue culture techniques for some plants are disclosed, for example, in US2004/091780 (Parvatam et al.), WO 2005/122752, U.S. Pat. No. 6,815,205, and WO 89/10958.

Unfortunately, identifying the various tissue culture conditions which result in successful propagation, can be extremely difficult and is mostly an empirical process. Although general tissue culture techniques have been known since 1950s, there is considerable difficulty in predicting the effects of plant growth regulators: this is because of the great differences in culture response between species, cultivars, and even plants of the same cultivar grown under different conditions. “Each type of plant grown requires specific culture conditions the development of which involves a great deal of time and effort.” See WO 2005/122752, p. 1, lines 19-23. The part of the plant that efficiently responds to culture conditions and the preparation of that part, the response to tissue culture medium, the type and amount of plant growth regulators, and growth conditions, and then the conditions for rooting and hardening of the plant, each of these differ enormously from plant species to species and variety to variety. “Thus, inventing conditions for efficient regeneration of plants requires developing specialized knowledge about a given plant.” See Parvatam et al., US 2004/091780, paragraphs 6-9.

Thus, the need remains for an effective tissue culture process for Hoodia plants.

### SUMMARY OF THE INVENTION

The present invention includes a process of propagating Hoodia plants, the process comprising:
(i) sterilizing a Hoodia explant;
(ii) placing the explant in contact with a multiplication medium comprising:
   (a) basal salts,
   (b) vitamins,
   (c) a carbohydrate source,
   (d) a cytokinin at a concentration of from about 2 μM to about 60 μM,
   (e) an auxin at a concentration of from about 0 μM to about 15 μM,
   provided that when the auxin is present at a concentration above about 10 μM, the cytokinin concentration is below about 44 μM; and
(iii) growing the Hoodia explant on the multiplication medium, to obtain at least one shoot on the explant.

The inventive process is an effective, commercially suitable alternative to growing Hoodia plants from seeds. The inventive process is based, in part, on the discovery that the type and relative amounts of cytokinin and auxin (when the latter is present) are critical in order to attain Hoodia plantlets with at least one new shoot, preferably 3 to 7 shoots, preferably with an average of at least 3 shoots, more preferably with an average of at least from 3 to 5 shoots, most preferably an average of 3.5 or 4 shoots per explant. The inventive process is most commercially feasible at the multiplication rate of at least 3 new average shoots per explant. Callus formation is preferably to be avoided or minimized, in order to maintain the genetic conformity of the regenerated plant to the mother plant. The number of new shoots is important, because each new shoot represents a potential new plant.

### DETAILED DESCRIPTION OF THE INVENTION

Except in the operating and comparative examples, or where otherwise explicitly indicated, all numbers in this description indicating amounts of material or conditions of reaction, physical properties of materials and/or use are to be understood as modified by the word “about.”

It should be noted that in specifying any range, any particular upper limit can be associated with any particular lower limit.

For the avoidance of doubt the word “comprising” is intended to mean “including” but not necessarily “consisting of” or “composed of.” In other words, the listed steps or options need not be exhaustive.

The term “shoot” means an organ of the Hoodia plant that grows above the soil surface. The shoot originates from the apical meristem plus one to several primordial leaves and also includes buds (small swelling) that will eventually develop into shoot.

“μM” means micromolar.

### Hoodia Plants

The invention is useful for propagating Hoodia plants. The Hoodia genus includes but is not limited to Hoodia gordonii, Hoodia currorii, Hoodia juttae, Hoodia dregei, Hoodia parviflora, Hoodia pilifera, Hoodia alstonii, Hoodia flava, Hoodia officinalis, Hoodia mossamedensis, Hoodia triebneri, Hoodia pedicellata, Hoodia rushii. The invention is particularly valuable for Hoodia gordonii plants, as the actives derived from Hoodia gordonii plants have optimum safety and efficacy for weight management products. The demand for appetite suppressant actives derived from the Hoodia plants and so, for Hoodia plants themselves, continues to grow.

### Explant

An explant (a piece) of the Hoodia plant is obtained, typically from a field or a greenhouse. Preferably, a shoot tip from a healthy, vigorous growing plant is obtained, preferably by cutting it off the mother plants. The length of the suitable explant piece is typically from 2 to 7 cm, preferably from 3 to 5 cm. The explants are then prepared for tissue culture medium as soon as possible, to avoid desiccation and build-up of exogenous contaminants (bacteria and fungus). The preparation steps include sterilization (preceded optionally by cleaning) and, optionally, de-spiking the explant. De-spiking involves carefully removing the spikes, 1 to 2 cm from the tower meristem e.g. with a surgical blade. It was found, as part of the present invention, that de-spiking of Hoodia explants results in a higher rate of introducing clean plants in the initiation medium because most of the explant tissues have a better contact with the sterilizing solution. The explants may be cleaned before sterilization. The cleaning is helpful if explants are obtained from the field; it is best done under running tap water for a few minutes, typically from 2 to 10 minutes, preferably from 3 to 5 minutes, so as to remove the debris from the field. The explants are then placed in sterile containers and sterilized. Suitable sterilization is by treatment of explants with mercuric chloride (0.1% solution), typically for 2 to 10 minutes, preferably from 3 to 6 minutes, followed by treatment with sodium hypochlorite solution (30% solution) for 25-40 minutes, more preferably for 20 to 30 minutes in order to obtain clean plants which also survive sterilization and are able to produce new shoots. The sterilized explants are placed into a multiplication medium. Preferably, the explants are placed into initiation medium prior to the multiplication stage, in order to ascertain that the plants are clean and free of bacterial or fungal growth. The initiation medium is of the same composition as the multiplication medium and may further comprise an antibiotic. The explants may typically be kept in the initiation medium for two to three weeks.

### Multiplication

According to the present invention, the type and the amount of the auxin and cytokinin in the multiplication medium is critical to promote the formation of at least one new shoot, preferably 3 to 7 shoots (with an average of at least from 3 to 5 shoots, more preferably an average of 3.5 to 4 shoots), white still avoiding or minimizing profuse callus formation at the base which interferes with shoot and root formation.

Suitable cytokinins are selected from adenine cytokinins and phenylurea cytokinins. Adenine cytokinin include but are not limited to kinetin, zeatin, and benzylaminopurine (BAP) (the latter also known as benzyladenine). Phenylurea cytokinin or substituted phenylurea is selected from the group consisting of N,N′-diphenylurea and thidiazuron (TDZ), and 6-(dimethylallyamino) purine (2iP).

The preferred cytokinins are BAP, kinetin, zeatin and 2iP because they are commercially available. Most preferably, cytokinin is selected from BAP and kinetin and optimally is BAP.

Auxin is preferably co-present, especially to obtain explants with 2 or more shoots. Suitable auxins include natural and synthetic auxins. Natural auxins include but are not limited to indole-3-acetic acid (IAA) and its conjugates which include but are not limited to IAA alanine, IAA phenylalanine, IAA aspartic acid, IAA inositol and IAA acetylglycine. Synthetic auxins include but are not limited to 2,4-dichlorophenoxyacetic acid (2,4-D), α-naphthalene acetic acid (NAA), 2-naphthyoxyacetic acid (NOA), 2-methoxy-3,6-dichlorobenzoic acid (dicamba), 4-amino-3,5,6-trichloropicolinic acid (tordon or picloram), and 2,4,5-trichlorophenoxyacetic acid (2,4,5-T).

The preferred auxins are IAA and its conjugates, e.g. IAA alanine, IAA aspartic acid, IAA inositol and IAA phenylalanine which have high activity and are cheaper to use in tissue culture. Most preferably, auxin is selected from IAA alanine and IAA, and optimally is IAA.

The amounts of auxins and cytokinins are as follows:

| | | | More | |
|---|---|---|---|---|
| | Generally | Preferably | Preferably | Optimally |
| Cytokinin | 2 to 60 μM | 4 μM to 45 μM | 9 μM to 25 μM | 17.74 μM |
| Auxin | 0 to 15 μM | 2 μM to 12 μM | 2 μM to 10 μM | 5.71 μM |

A basal mixture containing the micro and macro salts and vitamins is used for the multiplication medium. Suitable mixtures include but are not limited to Murashige & Skoog (ME&S) medium (Murashige and Skoog 1962), or a Gamborg B-5 medium containing the micro and macro salts and M&S vitamins (Gamborg et al. 1968). The preferred basal salt mixture is M&S medium, in order to minimize callus growth at the base of the shoots and some undesirable browning of the medium.

The multiplication medium also comprises a carbohydrate source, typically selected from sugar, starch and mixtures thereof. Carbohydrate source in the medium plays a major role in photosynthesis of the plants. Suitable sugars include but are not limited to sucrose, fructose, galactose, glucose, raffinose or maltose, more preferably sucrose and glucose and most preferably sucrose. The sugar is included typically in a concentration of from 10 to 40 g/l, preferably from 20 to 30 g/l and most preferably 30 g/l.

A preferred optional ingredient in the multiplication medium is gibberellin, in order to promote further shoot multiplication and also to promote shoot elongation. Longer shoots are preferable, because they are easier to cut off for further propagation and at the later rooting stages root induction is better with longer shoots.

Suitable gibberelin is selected from the group consisting of gibberellic acid (GA₃, GA₄ and GA₇) and mixtures thereof and more preferably GA₃ which is a combination of GA₄ and GA₇. There are about 80 different gibberellins of which these three are most useful in promoting plant elongation. Gibberelin (GA₃) is employed at a concentration ranging from 1 μM to 14 μM, preferably from 2 μM to 10 μM, more preferably from 3 μM to 8 μM, and most preferably at an optimum concentration of 5.77 μM. Gibberelin may be added to the multiplication medium just at the later cycles of multiplication, or may be present through all cycles of propagation.

Multiplication medium preferably includes a gelling agent. Suitable gelling agents include but are not limited to gelrite, agarose, agar, starch, gellan gum and preferably are selected from gelrite and agar and most preferably gelrite, because it's a clear solidifying agent which makes it easier to see whether there is bacterial or fungal growth. Plants may also be grown on liquid medium without any getting agents.

Other suitable ingredients include coconut water which can serve as a vitamin supplement, and other basal salt mixes.

The explants are placed 1-1.5 cm deep into the sterile solidified multiplication medium in an erect position.

It has been found, as part of the present invention, that the spacing of explants, i.e. the distance between explants, plays an important role in successful multiplication. Hoodia shoots grow sideways and diagonally upwards and thus need sufficient room for successful growth. Too much space between the explants is undesirable, however, as it leads to over-spending of the multiplication containers and the medium, resulting in less commercially attractive process. It has been found that the optimum distance between the explants is from 0.5 cm to 3 cm, preferably from 1 to 2, and optimally 1.2-1.5 cm.

The explants are kept in the multiplication medium for a 3 to 4 week interval per cycle; at 26±2° C. in a medium where the pH was adjusted to 5.8 and the tubs are placed in a growth room on a bench with 16/8 h light dark photoperiod (39-90 μE/m²/s) provided by cool white fluorescent tubes.

To build up the stock, new shoots that originate from the explant are cut off and are transferred on a monthly basis onto fresh multiplication medium for further multiplication. For commercial micropropagation, the propagation cycle is repeated for 3 to 5 cycles after initiation, i.e. for the total of four to six months. Rooting can be done at any of these phases depending on the need. For commercial production, it is preferred to root the plants after the fifth cycle.

For commercial tissue culture micropropagation of Hoodia according to the present invention, from one single explant, it is possible to have approximately 600 to 800 plants after 5 to 6 months in the multiplication medium.

The explants with new shoots are then transferred to a rooting medium for rooting, to obtain rooted plantlets.

It should be noted that various actives, including steroidal glycosides, may be extracted at any point during the inventive process, although their amount will be lower than in the mature plant, due to the size and maturity or age of the plant.

### Rooting

The rooting medium comprises the same basal salt mixture, vitamins, and phytohormones, as described above with respect to the multiplication medium, but at different preferred combinations and concentrations, in order to promote root formation and to avoid or minimise excessive callus growth at the cutting base. Preferably, a mixture of auxins is employed, especially IBA and NAA. The growth regulator, gibberelin, is included, most preferably GA3. The auxins and gibberelin are included according to the present invention typically in the following amounts:

| | Generally | Preferably | Most preferably |
|---|---|---|---|
| IBA | 10 to 50 μM | 25 to 36 μM | 29.50 μM |
| NAA | 3 to 11 μM | 5 to 11 μM | 5.37 μM |
| Gibberelin | 1 to 6 μM | 3 to 6 μM | 2.89 μM |

The rooting medium comprises the basal salt mixture at half strength concentration and vitamins at full strength concentration. The preferred basal salt mixture is M&S at half strength concentration.

The rooting medium preferably includes the same sugar and in the same concentration as described above for the initiation/multiplication medium.

The rooting medium preferably further comprises charcoal, in order to reduce callus growth. In addition, charcoal helped in leaching excess cytokinin and auxin in the media, allowing plants to start producing their natural rooting hormones in vitro. Charcoal is included in the concentration of from 0.5 to 4 g/l, more preferably from 1 to 3 g/l, most preferably from 1 to 2 g/l.

Preferably, gelrite is included in both the initiation/multiplication and the rooting medium to solidify the medium.

Preferably, an antibiotic is included, e.g. cefotaxime.

The explants are placed onto rooting medium 1-2 cm deep in the medium in an erect position. The distance between the explants is the same as for the multiplication phase, although they may be placed closer (i.e. about 0.5-1 cm apart), as they are no longer producing new shoots.

The rooted plantlets are kept in the rooting medium for 3-4 weeks, under conditions of darkness at temperature of 26±2° C. and later are placed under light for two to three weeks before going into the greenhouse for hardening.

It should be noted that additional phytohormones (auxins, cytokinins) may be present in the rooting and/or multiplication medium.

### Hardening

The hardening of the Hoodia plantlets is done, in order to acclimatize the plantlet to in-vivo (external) conditions. It has been found, as part of the present invention that the micropropagated rooted Hoodia plantlets do not harden welt under conditions that simulate wet dessert soil conditions, which may lead to vitrification (leaf or shoot having a glassy appearance) and fungal infestation. Rather, the plantlets are hardened best by using a gravel sort of “granite” mix, which allows for adequate drainage.

The plantlets are kept for hardening in a porous granitic fine mix and placed in a dry area with water being applied as a mist once in two days to avoid bacterial or fungal growth. This is done for three weeks until new true roots are formed before they can be transferred to the greenhouse where they receive minimal watering, twice a week for 3-6 weeks before going to the field.

Preferably, the plantlets are transferred to the field after gradual acclimatization. After the plants have established, which will normally take around 2 months, they should be placed for one hour under external conditions and the next day for two hours until when they become acclimatized before they can be transplanted to the field.

The following specific examples further illustrate the invention, but the invention is not limited thereto.

In all the multiplication and later rooting trials, the experiments were carried out in a randomised factorial design with at least four replicates per treatment. The student's t-test was used to determine significant difference between the phytohormone treatments. P values≦0.05 was considered significant.

### EXAMPLE 1

Example 1 investigated the effect of various sterilization protocols on the number of clean shoots and their ability to produce new shoots after sterilization.

A total of 211 shoot tips (5-6 cm) of fresh H. gordonii plant material were collected from cultivation sites.

The shoot tips were washed thoroughly for 3-5 minutes under running tap water and liquid detergent to remove any debris from the field. The spikes on some of the plants were carefully removed with a surgical blade before the sterilisation process. Clean shoot segments were trimmed and placed in a sterile tub containing 0.1% mercuric chloride solution. Each tub contained around 10 shoot tips placed in 300 ml of the sterilizing solution. The tubs were placed on a shaker at low speed 80 rpm for 5 minutes. After 5 minutes, the mercuric chloride was poured out and the plants were rinsed once in sterile distilled water. After rinsing with sterile distilled water, a second sterilisation process with 30% sodium hypochlorite solution was carried out. The tubs were placed on a shaker at a speed of 80 rpm for 30 or 40 minutes. A total of 188 shoots were sterilized for 30 minutes while 44 shoots were sterilized for 40 minutes. After draining the sodium hypochlorite, a batch of the plants were rinsed in distilled sterile water before putting in the initiation medium white another batch was placed directly into the initiation medium without rinsing. Recording of the percentage clean plants was done two weeks after initiation. Clean plants were shoots that were free of visible bacteria or fungal growth.

After sterilisation, the shoot tips were cultured on Murashige and Skoog (M&S) basal salt medium with M&S vitamins supplemented with 30 g/l sucrose, 3 g/l gelrite, 25 mg/l cefotaxime. The phytohormones used for the initiation medium were 17.74 μM Benzylaminopurine (BAP) and 5.71 μM Indole acetic acid (IAA). The pH of all the media tested was adjusted to 5.8 with either 1M potassium hydroxide (KOH) or 1N hydrochloric acid (HCl) before autoclaving at 121° C. for 20 minutes. Each 250 ml bottle contained one shoot tip. All the cultures were incubated at 26±2° C. in a growth room with a 16/8 hour light/dark photoperiod (39-90 μE/m²/s). New shoot growth normally started a week after sterilisation. After two weeks in culture, bottles that were visibly clean were counted. Each bottle had a single shoot tip explant, to avoid contamination.

TABLE 1
Percentage clean plants in relation to duration of sterilization

| | 40 minutes of |
|---|---|
| 30 minutes of sterilisation | sterilisation |
| 170 plants out of 188 | 41 plants out of 44 |
| plants 86.5% clean plants | total 92.7% clean plants |

In Table 1 above, 86.5% of the plants survived the 30 minutes sterilisation treatment, while 92.7% survived the 40 minutes treatment. Shoot tips that underwent the 40 minute treatment were heavily bleached on the external tissue whilst the 30 minute sterilised plants were still green. However, the shoots that were treated for 40 minutes still produced new shoots. The difference between the two time interval treatments was the time taken for shoots to emerge from plants treated with bleach for 40 minutes, there was about one week difference in favour of plants treated for 30 minutes.

There was no significant difference between shoots that were de-spiked before culturing or shoots that still had their spikes before culturing. If shoots are still clean and fresh, there is no need for de-spiking. However, for plants that have debris, it is advisable to remove the lower spikes before sterilisation. Removal of spikes has the disadvantage of building up phenolics in the medium. Phenolic build-up in the medium was drastically reduced after the first transfer onto fresh medium. The combined treatment of mercuric chloride and sodium hypochlorite was the reason for the high successful initiation percentage. The 0.1% mercuric chloride treatment mode of action is to inhibit microbial activity by combining cellular proteins containing the sulfhydryl groups. Sterilizing the shoots only with sodiumhypochloride resulted in high (80%) contamination rates from field grown cultures.

### EXAMPLE 2

Example 2 investigated the effect of basal salt medium on shoot multiplication. Two basal salt media (Murashige and Skoog and Gamborg B-5 medium—see Table 2) were tested for shoot multiplication.

TABLE 2
Murashige and Skoog (M&S) basal mixture
and Gamborg B-5 basal mixture.

| Component (mg/L) | M&S mixture | Gamborg B5 medium |
|---|---|---|
| Ammonium nitrate | 1650.0 | |
| Ammonium sulphate | | 134.0 |
| Boric acid | 6.2 | 3.0 |
| Calcium chloride anhydrous | 332.2 | 113.24 |
| Cobalt chloride.6H₂O | 0.025 | 0.025 |
| Cupric sulfate.5H₂O | 0.025 | 0.025 |
| Na₂-EDTA | 37.26 | 37.3 |
| Ferrous sulfate.7H₂O | 27.8 | 27.85 |
| Magnesium sulfate | 180.7 | 17.099 |
| Manganese sulfate.H₂O | 16.9 | 10.0 |
| Molybdic acid (sodium salt).2H₂O | 0.25 | 0.25 |
| Potassium iodide | 0.83 | 0.75 |
| Potassium nitrate | 1900.0 | 2500.00 |
| Potassium phosphate monobasic | 170.0 | |
| Sodium phosphate monobasic | | 130.5 |
| Zinc sulfate.7H₂O | 8.6 | 2.0 |
| myo-Inositol | 100.0 | 100.0 |
| Nictotinic acid (free acid) | 0.5 | 1.0 |
| Pyridoxine.HCl | 0.5 | 1.0 |
| Thiamine.HCl | 0.5 | 10.0 |

All the other supplements sucrose, gelrite and the vitamin mixture were the same as in the initiation medium in Example 1. Results on the multiplication rate of the shoots were obtained after two sub-cultures. Shoot proliferation was better in M&S medium when compared to Gamborg B5 medium. In Gamborg B5 medium, callus developed at the base of the shoots and furthermore, there was conspicuous browning of the growing medium at the plant base. This is a stress response by the plants. For further evaluations, M&S was used as the basal salt and vitamin mixture for all the experiments.

### EXAMPLE 3

This example tested the effect of different benzylaminopurine (BAP, cytokinin), indoleacetic acid (IAA, auxin) concentration and combinations on shoot multiplication.

The basal salt medium used for this experiment was M&S basal salt and vitamins. All the other components were the same as used for initiation medium in Example 1. The cytokinin BAP was used at four different concentrations (0, 11.09, 22.19 and 44.38 μM) and the auxin IAA was used at four different concentrations (0, 5.71, 11.42 and 22.84 μM). Factorial experiments were designed to test the effect of these phytohormones on shoot multiplication.

Each treatment had four replicates. Results were obtained after two sub-cultures.

TABLE 3

| | | | Average | |
|---|---|---|---|---|
| | | | number of | |
| Experiment | IAA (μM) | BAP (μM) | new shoots | Observations |
| 1 | 0 | 11.09 | 2 | New shoot tip growth and |
| | | | | moderate side shoot production |
| 2 | 0 | 22.19 | 2 | New shoot tip growth and |
| | | | | moderate side shoot production |
| 3 | 0 | 44.38 | 1 | New shoot tip growth and |
| | | | | moderate side shoot production |
| 4 | 5.71 | 11.09 | 2 | Moderate side shoot |
| | | | | production and callus at shoot base |
| 5 | 5.71 | 22.19 | 4 | Most side shoots produced; |
| | | | | callus at base |
| 6 | 5.71 | 44.38 | 1 | Poor side shoot formation |
| 7 | 11.42 | 11.09 | 1 | High callus formation |
| 8 | 11.42 | 22.19 | 1 | Callus formation |
| A | 0 | 0 | 0 | No growth. Flowering observed |
| B | 5.71 | 0 | 0 | Plant growth, but no new |
| | | | | side shoots |
| C | 11.42 | 0 | 0 | Flowering. Callus at base of shoot |
| D | 11.42 | 44.38 | 0 | Callus formation |
| E | 22.84 | 0 | 0 | High callus formation |
| F | 22.84 | 11.09 | 1 (very | High callus formation |
| | | | poor | |
| | | | quality) | |
| G | 22.84 | 22.19 | 0 | High callus formation |
| H | 22.84 | 44.38 | 0 | High callus formation |

From the results in Table 3 above, it can be seen that the best cytokinin and auxin combination was BAP at 22.19 μM and IAA at 5.71 μM. An average of four side shoots per explant was observed using this hormone combination and concentration.

At concentrations higher than 44.38 μM of BAP, auxiliary shoots production was retarded and callus growth from the base of the shoots was quite visible. Higher concentrations of both the cytokinin and auxins inhibited growth. Since BAP at a concentration of 22.19 μM and the IAA (auxin) at 5.71 μM still produced some callus at the base it was decided to reduce the concentration of to BAP to 17.74 μM. At 17.74 μM, multiple shoots were still being produced on the initial explants and an average of 4 shoots per explant was observed with less callus growth.

### EXAMPLE 4

This example investigated the effect of spacing between the plants on shoot multiplication rate. For commercial application of this technology, the spacing between the shoots is vital. Shoot tips were trimmed at the base and cultured in tubs with the optimum multiplication medium. The number of shoots per tub varied at 5, 10, and 15 plants/tub, equivalent to 1.2, 1.5, and 2 cm spacing between the plants. Experiments were carried out in triplicate sets and the results recorded were (a) number of side shoots produced per tub which is the multiplication rate and (b) shoot elongation dependent on number of shoots cultured per vessel. The results indicated that the optimum spacing between the plants is 1.2 cm at the first and second cycle and the spacing could be decreased to 0.75-1 cm in the third and fourth cycle, at which stage the side shoot plant size is smaller.

### EXAMPLE 5

This Example investigated the effect of various giberellic acid concentrations on shoot elongation and proliferation.

TABLE 4
Effect of gibberillic acid on shoot proliferation (rep = replicates)

| | Number of new shoots | | | | | | |
|---|---|---|---|---|---|---|---|
| GA₃ (μM) | Rep1 | Rep 2 | Rep 3 | Rep 4 | Rep 5 | Rep 6 | Rep 7 |
| 1.44 | 1 | 2 | 0 | 0 | 1 | 2 | 1 |
| 2.89 | 3 | 2 | 2 | 3 | 4 | 4 | 3 |
| 5.77 | 4 | 5 | 5 | 4 | 6 | 7 | 8 |

Results of the experiment show that at the concentration of 2.89 μM and 5.77 μM of GA₃, the shoots were elongated and multiplied better. GA₃ at 5.77 μM showed an increase in length of ±1 cm of the initial explant. An average of four shoots per explant was observed when the GA₃ concentration was increased from 2.89 μM to 5.78 μM with 17.74 μM of BAP and 5.71 μM of IAA. As the concentration increased, the shoot length was retarded but side shoots were formed.

### EXAMPLE 6

Example 6 tested the effect of three sucrose concentrations 0, 20 and 30 g/l on new shoot formation. All the other supplements were the same as for the initiation medium in Example 1. M&S basal salt and vitamin was used as the basal medium.

The sucrose concentrations used for the multiplication experiments were 0, 20 and 30 g/l respectively. There was no multiplication on plants placed in the control (0 g/l) medium which had no sugar. The shoots were stunted and started to turn to yellow after three weeks in culture. Lowering of the sucrose concentration from 30 g/l to 20 g/l in the multiplication medium had no significant effect on shoot development and multiplication.

While the present invention has been described herein with some specificity, and with reference to certain preferred embodiments thereof, those of ordinary skill in the art will recognize numerous variations, modifications and substitutions of that which has been described which can be made, and which are within the scope and spirit of the invention. It is intended that all of these modifications and variations be within the scope of the present invention as described and claimed herein, and that the inventions be limited only by the scope of the claims which follow, and that such claims be interpreted as broadly as is reasonable.

What is claimed is:

1. A process of propagating Hoodia plants, the process comprising:
(i) sterilizing a Hoodia explant;
(ii) placing the explant in contact with a multiplication medium comprising:
(a) basal salts,
(b) vitamins,
(c) a carbohydrate source,
(d) a cytokinin at a concentration of from about 2 μM to about 60 μM,
(e) an auxin at a concentration of from about 0 μM to about 15 μM,
provided that when the auxin is present at a concentration above about 10 μM, the cytokinin concentration is below about 44 μM; and
(iii) growing the Hoodia explant on the multiplication medium, to obtain at least one shoot on the explant.

2. The process of claim 1 wherein the Hoodia plant is selected from Hoodia gordonii, Hoodia currorii, Hoodia juttae, Hoodia dregei, Hoodia parviflora, Hoodia pilifera, Hoodia alstonii, Hoodia flava, Hoodia officinalis, Hoodia mossamedensis, Hoodia triebneri, Hoodia pedicellata, Hoodia rushii.

3. The process of claim 1 wherein the Hoodia plant is Hoodia gordonii.

4. The process of claim 1 wherein the auxin is present in the multiplication medium at a concentration from about 2 μM to about 12 μM.

5. The process of claim 1 wherein the auxin is selected from the group consisting of natural auxin and synthetic auxin.

6. The process of claim 5 wherein the auxin is a natural auxin and is selected from the group consisting of indole acetic acid (IAA), IAA alanine, IAA phenylalanine, IAA aspartic acid IAA acetylglycine and mixtures thereof.

7. The process of claim 5 wherein the auxin is a synthetic auxin and is selected from the group consisting of 2,4-dichlorophenoxyacetic acid (2,4-D), α-naphthalene acetic acid (NAA), 2-methoxy-3,6-dichlorobenzoic acid (dicamba), 4-amino-3,5,6-trichloropicolinic acid (tordon or picloram), and 2,4,5-trichlorophenoxyacetic acid (2,4,5-T).

8. The process of claim 1 wherein the cytokinin is present in the multiplication medium at a concentration of from about 9 μM to about 25 μM.

9. The process of claim 1 wherein the cytokinin is selected from the group consisting of adenine cytokinins and phenylurea cytokinins.

10. The process of claim 9 wherein the cytokinin is an adenine cytokinin and is selected from the group consisting of kinetin, zeatin, and benzylaminopurine (BAP) or benzyladenine (BA).

11. The process of claim 9 wherein the cytokinin is a phenylurea cytokinin or substituted phenylurea and is selected from the group consisting of N,N′-diphenylurea and thidiazuron (TDZ).

12. The process of claim 1 wherein the auxin is indole acetic acid and it is present in the multiplication medium at a concentration of about 5.71 μM and the cytokinin is benzylaminopurine and it is present in the multiplication medium at a concentration of about 17.74 μM.

13. The process claim 1 wherein the explants are placed onto the multiplication medium at the distance from each other of from about 0.5 cm to 3 cm.

14. The process of claim 1 wherein the multiplication medium further comprises a gibberellin at a concentration of from about 1 μM to about 14 μM.

15. The process of claim 14 wherein the gibberellin is gibberellic acid.

16. The process of claim 1 wherein the basal salts and vitamins are delivered to the medium by Murashige & Skoog mixture.

17. The process of claim 1 wherein the carbohydrate source is a sugar selected from the group consisting of sucrose, fructose, galactose, glucose, raffinose, maltose and mixtures thereof.

18. The process of claim 17 wherein the sugar concentration is from about 10 to about 40 g·L⁻¹.

19. The process of claim 1 further comprising the step of de-spiking the explant prior to placing the explant into the multiplication medium.

20. The process of claim 1 wherein the sterilization is by combined treatment of mercuric chloride and sodium hypochlorite.

21. The process of claim 1 further comprising transferring the explant with shoots into a rooting medium to obtain rooted plantlets.

22. The process of claim 21 further comprising hardening the rooted plants.

23. The process of claim 1, further comprising obtaining further explants from the explants with new shoots and subjecting the further explants to a second cycle of multiplication by repeating step (iii), to obtain further explants with new shoots.

24. The process of claim 23 wherein the multiplication cycle is repeated from 4 to 6 times.

25. The process of claim 1 wherein the resulting explant has an average of at least 3 shoots.
